(19) United States
(12) Patent Application Publication
(10) Pub. No.: US 2008/0221033 A1
(43) Pub. Date: Sep. 11, 2008

(54) Monitoring of Cardiac Natriuretic Peptides During Diagnosis, Management, and Treatment of Cardiac Diseases

(75) Inventors: Jens-Peter Seher, Stuttgart (DE); Malcolm G. Pluskal, Acton, MA (US)

Correspondence Address: PHILIPS INTELLECTUAL PROPERTY & STANDARDS, 595 MINER ROAD, CLEVELAND, OH, 44143, US

(73) Assignee: Koninklijke Philips Electronics N. V., Eindhoven (NL)

(21) Appl. No.: 11/996,810

(22) PCT Filed: Jul. 27, 2006
(86) PCT No.: PCT/IB06/52578

§ 371 Date: Jan. 25, 2008

Related U.S. Application Data
(60) Provisional application No. 60/703,928, filed on Jul. 29, 2005.

(51) Int. Cl. G01N 33/68; A61K 38/00; A61P 9/00; A61B 5/00

(52) U.S. Cl. 514/12; 436/34; 600/309

(57) ABSTRACT

In a cardiac monitoring method, at east an inactive natriuretic peptide (22) and an active natriuretic peptide (20) are assayed in a biological sample taken from a patient. Both the inactive and active natriuretic peptides are produced in the patient responsive to cardiac stress. A therapeutic agent (26), such as a synthetic or mimetic natriuretic peptide, is administered to the patient. The administered therapeutic agent substantially interferes with the active natriuretic peptide assay but does not substantially interfere with the inactive natriuretic peptide assay. Kinetics of the administered therapeutic agent are determined based on at least the active natriuretic peptide assay. Cardiac diagnostic information is determined based on at least the inactive natriuretic peptide assay.

The following relates to the medical diagnosis and treatment arts. It finds particular application in monitoring the diagnosis, management, and treatment of cardiac diseases based on levels of natriuretic peptides, and will be described with particular reference thereto. However, it also finds application in monitoring of cardiac diseases based on levels of various other cardiac markers.

According to the World Health Organization (WHO), the diagnosis for cardiac diseases is based on the presence of at least 2 of the following criteria: An electrocardiogram (ECG), patient history and serum cardiac markers. The 4 classical serum markers are: Total-CK (Creatine Kinase), CK-MB (Creatine Kinase MB isoenzyme), Myoglobin and cardiac Troponins. In the last years the natriuretic peptides have gained more and more interest as serum cardiac markers. A significant advance in the diagnosis and monitoring of cardiac diseases has been the development of blood automated and semi-automated assay tests that measure the level of a cardiac marker in a blood sample drawn from a patient known or suspected to be suffering from a cardiac disease. Some cardiac markers of interest include: atrial natriuretic peptide (ANP); N-terminal proANP (NT-proANP); brain natriuretic peptide (BNP); N-terminal pro-BNP (NT-proBNP); C-type natriuretic peptide (CNP); and so forth. These various cardiac markers are secreted by the patients body during cardiac stress, and hence correlate with cardiac stress. During a heart attack, the level of cardiac markers typically increases sharply, and then decreases as the cardiac marker is utilized, metabolized or otherwise removed from the bloodstream. In the case of a congestive heart failure or other chronic cardiac stresses, a persistent level of such cardiac markers may be observed in the blood. Thus, the assayed level of cardiac marker is useful in detecting the presence of heart failure, determining its severity, and estimating prognosis. By monitoring cardiac marker levels in blood serum during treatment, the effectiveness of the treatment can be assessed.

Some of these cardiac markers have also been considered as possible therapeutic agents for treating cardiac diseases. Scios, Inc., for example, developed Natrecor® (nesiritide), a recombinant form of human B-type natriuretic peptide (hBNP), which was approved by the Food and Drug Administration (FDA) in 2001 for the intravenous treatment of patients with certain types of acute heart failure. Synthetic therapeutic agents, such as nesiritide, which correspond to cardiac markers have shown substantial promise and demonstrated clinical success. In patients hospitalized with congestive heart failure, nesiritide reduces pulmonary capillary wedge pressure, pulmonary arterial pressure, right atrial pressure and systemic vascular resistance, all resulting in clinical improvement. Nesiritide has certain advantages over parental vasodilators (e.g. nitroprusside and nitroglycerin) and inotropic agents (e.g. dobutamine and milrinone) currently used in treating decompensated heart failure, being more effective and better tolerated, allowing patients to continue their usual medications while in the hospital, and not automatically requiring invasive monitoring. The terminal half-life of nesiritide in the bloodstream is 20 minutes, the onset of action is 15 min, and a fixed dose of nesiritide has a sustained effect over 24 hours.

However, a therapeutic agent which is a synthetic form of a cardiac marker or is otherwise mimetic of a cardiac marker typically interferes with the blood assay of the natural cardiac marker. For example, nesiritide is typically detected by the same blood assay techniques used to monitor the level of natural BNP. Accordingly, the BNP cardiac marker assay cannot separately monitor both the progression of heart failure (diagnostic aspect) and the efficiency of therapy using a synthetic form of the cardiac marker (kinetic aspect). It is desirable to monitoring both the kinetics of the administered therapeutic agent, and the progression of the underlying heart disease, in order to assess the effectiveness of the treatment.

According to one aspect, a cardiac monitoring method is disclosed. At least an inactive natriuretic peptide and an active natriuretic peptide are assayed in a biological sample taken from a patient. Both the inactive and active natriuretic peptides are produced in the patient responsive to cardiac stress. A therapeutic agent is administered to the patient. The administered therapeutic agent substantially interferes with the active natriuretic peptide assay but does not substantially interfere with the inactive natriuretic peptide assay. Kinetics of the administered therapeutic agent is determined based on at least the active natriuretic peptide assay. Cardiac diagnostic information is determined based on at least the inactive natriuretic peptide assay.

According to another aspect, a cardiac monitoring method is disclosed. A therapeutic agent is administered to a patient. A first cardiac marker assay is performed that is substantially affected by the therapeutic agent. A second cardiac marker assay is performed that is substantially not affected by the therapeutic agent. Kinetics of the administered therapeutic agent is determined based on at least the first cardiac marker assay. Cardiac diagnostic information is assessed based on at least the second cardiac marker assay.

According to another aspect a cardiac monitoring apparatus is disclosed. A blood extraction device is adapted to extract a plurality of blood samples from a patient undergoing treatment including administration of a synthetic or mimetic form of an active natriuretic peptide. An assay device is adapted to perform a blood assay yielding at least (i) an active natriuretic peptide level of a naturally produced natriuretic peptide and the synthetic or mimetic peptide and (ii) an inactive natriuretic peptide level for each blood sample. An interpretation device outputs at least one of (i) kinetics of the administered synthetic or mimetic natriuretic peptide based on at least the first cardiac marker assay, and (ii) cardiac diagnosis information based on at least the second cardiac marker assay.

One advantage resides in providing both kinetic and diagnostic information during treatment of a cardiac disease using a therapeutic agent that interferes with cardiac marker monitoring.

Another advantage resides in facilitating treatment of cardiac disease using nesiritide or other natriuretic peptide therapeutic agents.

Another advantage resides in extracting both uptake of a therapeutic agent and cardiac diagnostic information from a common blood assay.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

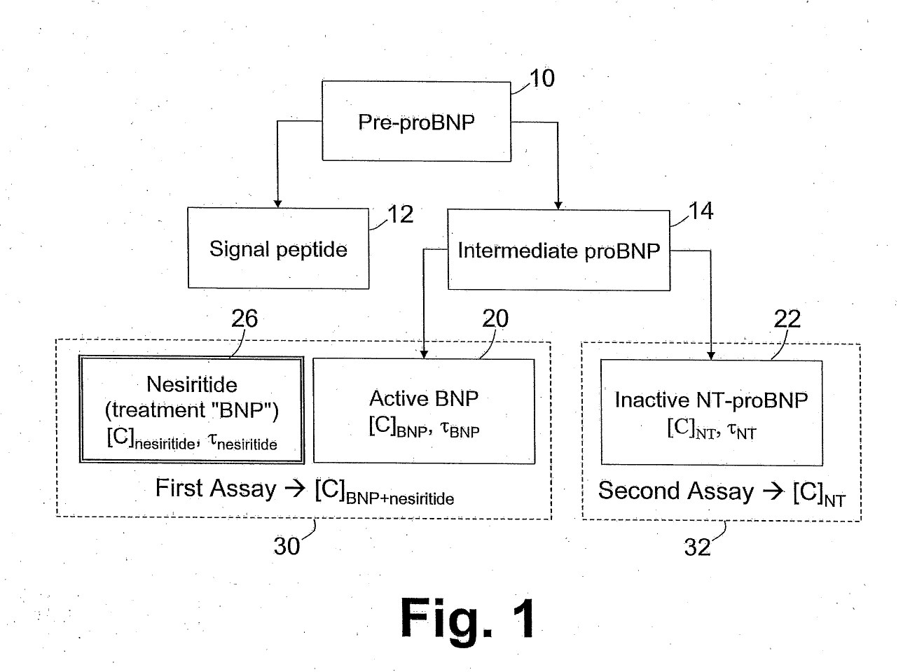

FIG. 1 diagrammatically shows (i) one possible biochemical pathway for generating active BNP and inactive NT-proBNP, (ii) administered nesiritide, (iii) a first assays that measures a combination of the BNP and nesiritide levels, and (iv) a second assay that measures the level of NT-proBNP.

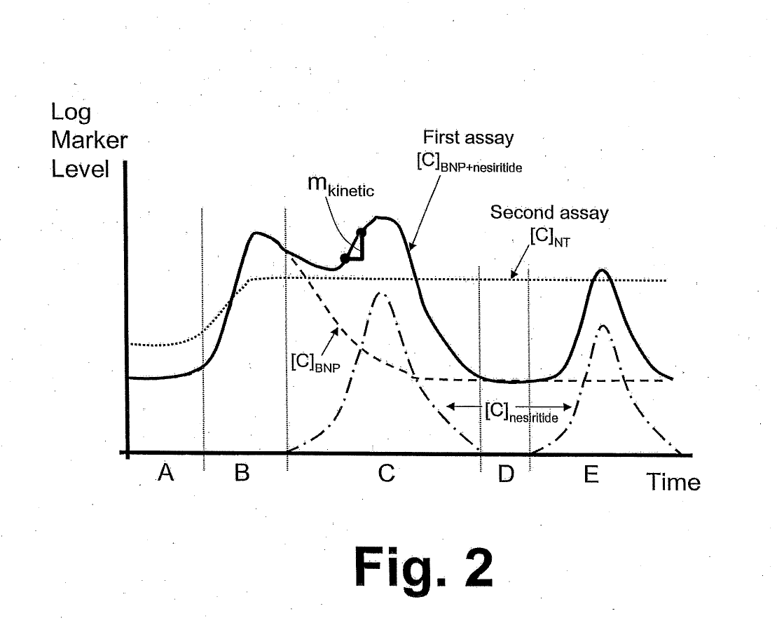

FIG. 2 diagrammatically plots the marker and assay levels of FIG. 1 for the case of a cardiac event such as a heart attack.

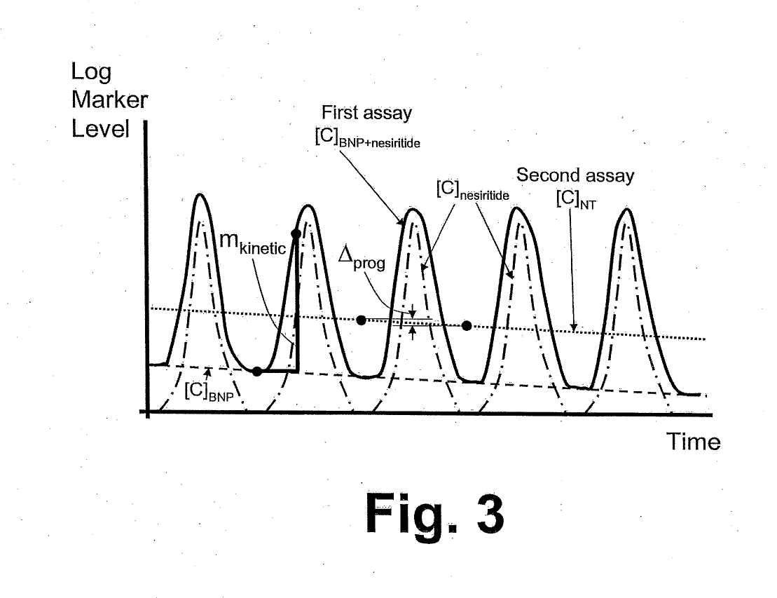

FIG. 3 diagrammatically plots the marker and assay levels of FIG. 1 for the case of treatment of chronic heart disease using a plurality of nesiritide treatments administered over a treatment period.

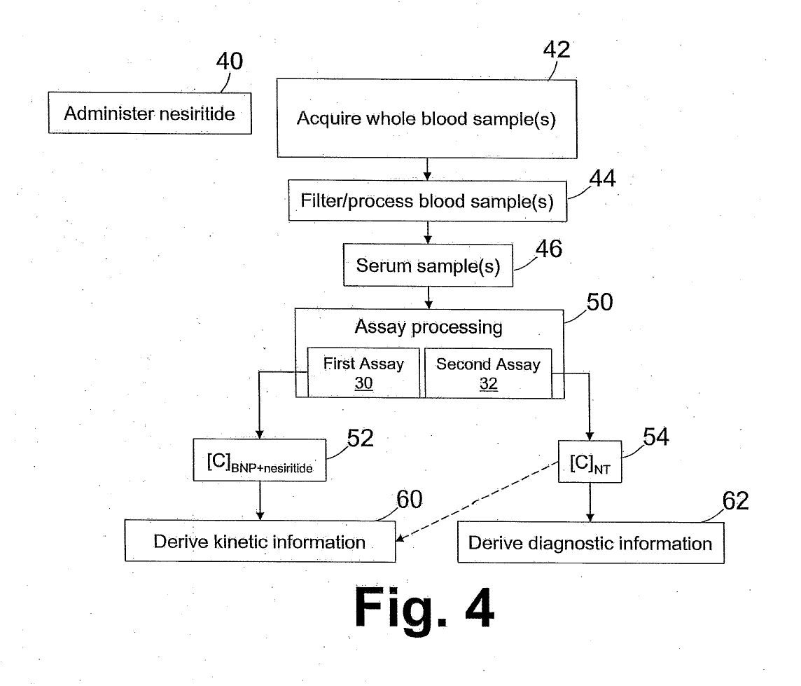

FIG. 4 diagrammatically shows a suitable cardiac monitoring method for monitoring both nesiritide kinetics and cardiac disease progression.

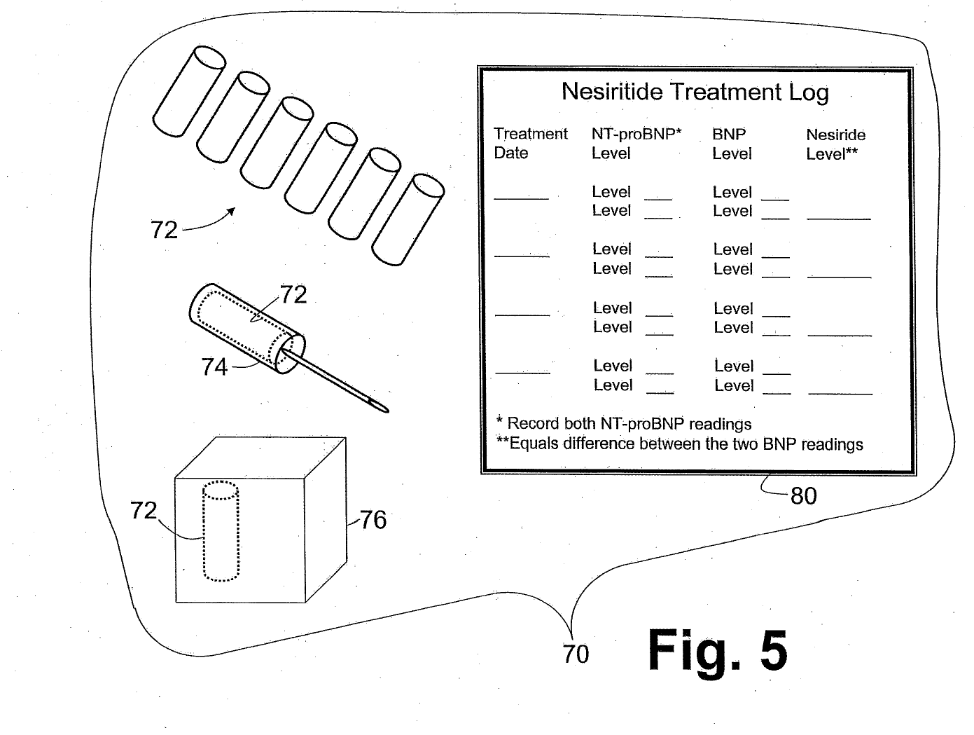

FIG. 5 diagrammatically shows a suitable cardiac monitoring apparatus for performing the cardiac monitoring method of FIG. 4.

With reference to FIG. 1, it is believed that the release or secretion of cardiac markers typically involves a series of biochemical reactions. Without being limited to any particular theory of operation, FIG. 1 illustrates what is believed to be a typical series of biochemical reactions resulting in the release or secretion of active BNP from myocardial cells. A precursor pre-proBNP protein 10 typically having 134 amino acids is cleaved into a signal peptide 12 typically having 26 amino acids, and an intermediate proBNP protein 14 typically having 108 amino acids. The intermediate proBNP 14 may in tam be cleaved into a physiologically active form of BNP 20, and a physiologically inactive form of NT-proBNP 22.

The level of active BNP 20 in the blood is denoted herein as [C]_{BNP}. The level of inactive NT-proBNP 22 in the blood is denoted herein as [C]_{NT}. The levels of BNP 20 and NT-proBNP 22 are typically related. It is believed that this relationship is due to the production of BNP 20 and NT-proBNP 22 from a common biochemical pathway such that they are produced in equimolar amounts; however, the relationship between the levels of BNP 20 and NT-proBNP 22 may be due to other factors such as secretion responsive to a common cardiac stressor.

Although the blood serum levels of BNP 20 and NT-proBNP 22 are typically related, the physiologically inactive NT-proBNP 22 is relatively more stable and stays resident longer in the blood compared with the physiologically active BNP 20. The terminal half-life of BNP 20 in the bloodstream is denoted herein as τ_{BNP}, and is believed to be typically of order of a few minutes to a few hours. In contrast, the terminal half-life of NT-proBNP 22 in the bloodstream, denoted herein as τ_{NT}, is typically substantially longer, and is believed to be about a month or longer.

The BNP 20 and NT-proBNP 22 cardiac markers are an example pair of related cardiac markers. It is expected that other pairs of cardiac markers may be similarly produced by related biochemical reactions or otherwise have related concentrations. Typically, if a common biochemical pathway or precursor molecule produces both an active natriuretic peptide or other cardiac marker and an inactive natriuretic peptide or other cardiac marker, then the terminal half-life of the inactive cardiac marker in the bloodstream will generally be substantially longer than the terminal half-life of the active cardiac marker in the bloodstream. It is expected that the inactive cardiac marker will typically have a terminal half-life at least ten times longer than the half-life of the active cardiac marker in the bloodstream, although the half-life difference may be less. For example, it is believed that ANP and NT-proANP are another related pair of active and inactive cardiac markers, respectively, derived from a common precursor molecule and hence generated in equimolar amounts. The terminal half-life of ANP in the bloodstream is believed to be substantially longer than the terminal half-life of NT-proANP in the bloodstream.

A physiologically active cardiac marker (BNP 20 in example FIG. 1, or ANP as another example) is a candidate for use as a treatment. For example, nesiritide (available from Scios, Inc. under the trade name Natrecor®) is a synthetic form of BNP that has been approved by the FDA for treatment of certain types of cardiac disease. Treatment agents that are synthetic ANP or mimetic of ANP are also of interest for treating arterial hypertension, congestive heart failure, and so forth.

Nesiritide 26 is used as a treatment agent in the example of FIG. 1. Other treatment agents that are mimetic of active BNP 20 may also be used. A first blood assay 30 is performed to assay the level of BNP 20. However, because nesiritide is typically detected by the same blood assay techniques used to monitor the level of natural BNP, the first assay 30 actually produces a level [C]_{BNP+nesiritide} which is indicative of a combination of the level [C]_{BNP} of natural BNP 20 in the blood serum and a level [C]_{nesiritide} of synthetic nesiritide 26 in the blood serum. Accordingly, the first assay 30 cannot provide, by itself, information on either the kinetics of the administered nesiritide 26 or the progression of the underlying heart disease indicated by the concentration [C]_{BNP} of natural BNP 20.

Accordingly, a second blood serum assay 32 is performed to assay the level of NT-proBNP 22. The second assay 32 is not substantially affected by the administered nesiritide 26; accordingly, the second assay 32 can be used to determine cardiac diagnostic information. The first assay 30 can then be used to determine kinetics of the administered nesiritide 26.

With reference to FIG. 2, the monitoring provided by the first and second assays 30, 32 is illustrated in the case of a cardiac event such as a heart attack. Various regions of FIG. 2 are denoted by letters “A”, “B”, “C”, “D”, and “E”. The region “A” preceding the cardiac event exhibits persistent levels of BNP and NT-proBNP due to an underlying cardiac disease. At a transition from region “A” to region “B”, the cardiac event occurs, causing cardiac stress. Responsive to the cardiac stress, the level [C]_{BNP} of BNP and the level [C]_{NT} of NT-proBNP both rise; however, by the end of region “B” the level of BNP begins to decrease due to its short terminal half-life in the bloodstream, and by the middle of the region “C” the level of BNP has returned to its persistent level indicative of the underlying cardiac disease, where it remains in regions “D” and “E”. In contrast, the level of NT-proBNP remains elevated throughout regions “C”, “D”, and “E” due to its substantially longer half-life in the bloodstream. At the transition from region “B” to region “C” a dose of nesiritide administered, producing a transient level [C]_{nesiritide} of nesiritide in the blood.

The first assay 30 outputs a level [C]_{BNP+nesiritide} indicative of the combination of the level of nesiritide and the level of natural BNP. The second assay 32 outputs a level [C]_{NT} indicative of only the level of NT-proBNP in the blood. The second assay is suitably used to determine cardiac diagnostic information, such as to estimate the severity of the cardiac event. The first assay 30 is suitably used to provide kinetics on the nesiritide treatment. For example, a difference or slope m_{kinetics} between two repetitions of the first assay 30 can be used to determine uptake of the nesiritide into the patient's bloodstream. In the region “D”, the nesiritide dose has dissipated from the bloodstream, and the patient has returned to the persistent levels of BNP and NT-proBNP existing prior to the cardiac event. In the region “E”, a follow-up dose of nesiritide is administered.

With reference to FIG. 3, the monitoring provided by the first and second assays 30, 32 is illustrated in the case of the nesiritide being administered a plurality of times (e.g., daily, every third day, or so forth) over a treatment period (e.g., one-week, one-month, indefinitely). Each administration of nesiritide produces a transient level [C]_{nesiritide} in the bloodstream. The first assay 30 can be used to assess uptake of the nesiritide by, for example, taking a first repetition of the first assay 30 just before administering the nesiritide, and a second repetition of the first assay 30 a fixed time interval after administering the nesiritide. The difference in [C]_{BNP+nesiritide} level measured by the first and second repetitions is a measure of the uptake of the nesiritide. The terminal half-life of nesiritide in the bloodstream is typically about 20 minutes, while the onset of therapeutic action is about 15 minutes, and the therapeutic action is sustained over about 24 hours in typical cardiac patients. The first and second repetitions can be separated by, for example, about 2-10 minutes.

While the first assay 30 provides kinetic information about the administered treatment agent 26, the second assay 32 provides diagnostic information about the progression of the cardiac disease. A difference Δ_{prog} between the level assayed using the second assay 32 on successive treatment dates shows, in the example FIG. 3, that the persistent level of NT-proBNP is decreasing, presumably responsive to the nesiritide therapy, indicating improvement in symptoms of the underlying cardiac disease condition.

With reference to FIG. 4, a suitable method for cardiac monitoring is described. In a treatment operation 40, nesiritide or another cardiac marker-mimetic therapeutic agent is administered. One or more blood samples are drawn in an operation 42. The blood samples can be drawn just prior to administering the nesiritide and/or just after administering the nesiritide. Typically, at least two blood samples are drawn to provide kinetic information about the nesiritide treatment. In a filtering process 44, the white and red blood cells are substantially removed from the blood sample to produce a blood serum sample 46. Assay processing 50 is performed to implement the first and second assays 30, 32. It is to be appreciated that typically both the first and second assays 30, 32 can be performed using a single blood serum sample 46. The first assay 30 provides the level [C]_{BNP+nesiritide} 52, while the second assay 32 provides the level [C]_{NT} 54. The level [C]_{BNP+nesiritide} 52 from two or more blood samples is used to determine kinetic information 60 about the administration 40 of nesiritide. The level [C]_{NT} 54 is used to determine diagnostic information 62 about the progression of the underlying cardiac disease.

In FIGS. 2 and 3, the kinetic information 60 is derived as a simple difference between the level [C]_{BNP+nesiritide} 52 measured from two successive blood samples. In other embodiments, a more elaborate derivation of kinetic information 60 may be performed. For example, because the levels [C]_{BNP} and [C]_{NT} are related, the level [C]_{BNP} may be estimated from the level [C]_{NT} assayed using the second assay 32. Then, the level [C]_{BNP+nesiritide} 52 assayed using the first assay 30 may be connected to remove the estimated level [C]_{BNP}, yielding the level [C]_{nesiritide}. In this approach, the level [C]_{nesiritide} of nesiritide may be assayable using only a single blood sample. In FIG. 4, the contemplated use of the level [C]_{NT} in determining or refining the kinetic information is indicated by a connection arrow drawn in phantom connecting the level [C]_{NT} 54 with the kinetic information determination 60.

With reference to FIG. 5, the described cardiac monitoring method advantageously employs existing techniques for assaying cardiac markers. Accordingly, a cardiac monitoring apparatus 70 is readily constructed, including a plurality of blood sample vials 72, cartridges, or so forth, and a blood extraction device 74 (such as a hypodermic needle) adapted to sequentially extract blood samples into the blood sample vials 72. An assay device 76 sequentially receives each filled blood sample vial 72 and performs standard BNP and NT-proBNP assays. The standard BNP assay corresponds to the first assay 30, and actually yields a level [C]_{BNP+nesiritide} indicative of a combination of the active natural BNP 20 and synthetic nesiritide 26 levels. The standard NT-proBNP assay corresponds to the second assay 32 and yields the NT-proBNP level [C]_{NT}. An interpretation device 80 outputs the kinetics information and the diagnostic information.

In the illustrated cardiac monitoring apparatus 70, the interpretation device 80 includes a “Nesiritide Treatment Log” card or display, on which for each date two sequential blood sample assays are recorded for each nesiritide treatment date over a treatment period. The two blood samples can be drawn before and a predetermined time after the nesiritide is administered, or can be drawn at two different predetermined times after the nesiritide is administered. By reading down the “NT-proBNP” column for successive treatment dates, the progression of the underlying cardiac disease can be ascertained. For example, if the recorded [C]_{NT} levels decrease over the course of the treatment period, this is an indication that the underlying cardiac disease is becoming less symptomatic. On the other hand, constant or increasing recorded [C]_{NT} levels suggest that the nesiritide treatment is ineffective. To derive the kinetic information on nesiritide uptake, the difference between the first assay 30 levels for the two blood samples drawn on a given treatment date is recorded. This difference is indicative of the uptake of nesiritide into the bloodstream.

Rather than a paper “Nesiritide Treatment Log” card 80, an electronic log can be maintained. For example, the “Nesiritide Treatment Log” card 80 can be an electronic spreadsheet that includes a processor that automatically performs the difference computation to determine the nesiritide level. Moreover, such an electronic spreadsheet optionally includes a more complex kinetic information derivation, for example taking into account the NT-proBNP level [C]_{NT} assayed using the second assay 32. Optionally, the processor analyzes the assay results over time to provide alarms or warnings, proposed diagnoses, proposed nesiritide dosage adjustments, or so forth.

Because the disclosed cardiac monitoring methods and apparatuses employ standard blood assay techniques, the disclosed cardiac monitoring apparatus can be embodied as a near-patient monitoring device in a hospital, for example in an emergency room. Similarly, the disclosed cardiac monitoring apparatus can be embodied as a test station or apparatus located in a physician's office or laboratory. It is also contemplated to employ the disclosed cardiac monitoring apparatus as an in-home testing device for self-testing performed by the patient. For example, the patient can have the vials 72 and blood-drawing device 74 at home, and can draw blood samples at appropriate times. The blood samples are then sent to a laboratory which has the assay device 76 and interpretation device 80. It is also contemplated to assay body fluids other than blood which may contain BNP, NT-proBNP, or other cardiac markers.

The illustrated methods and apparatuses using the combination of BNP and NT-proBNP assays to determine both kinetic and diagnostic information are examples. In other embodiments, for example, the first assay may monitor ANP, while the second assay may monitor NT-proANP. In other embodiments, the first assay may monitor CNP, while the second assay may monitor NT-proCNP. In other embodiments, the first assay may monitor DNP, while the second assay may monitor NT-proDNP. Still further, it is contemplated to monitor active and inactive cardiac markers derived from different biochemical pathways or precursor molecules. For example, monitoring of BNP and NT-proANP is contemplated. In this embodiment, BNP monitoring provides kinetic information on nesiritide uptake, while NT-proANP monitoring provides diagnostic information. Although BNP and NT-proANP are believed to be derived from separate biochemical pathways (and hence are not expected to be produced in equimolar amounts), both BNP and NT-proANP are believed to be produced in the patient predominantly due to cardiac stress; accordingly, monitoring both BNP and NT-proANP is expected to provide both kinetic and diagnostic information. Other assay combinations are also contemplated.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A cardiac monitoring method comprising:
assaying at least an inactive natriuretic peptide and an active natriuretic peptide in a biological sample taken from a patient, both the inactive and active natriuretic peptides being produced in the patient responsive to cardiac stress;
administering a therapeutic agent to the patient, the administered therapeutic agent substantially interfering with the active natriuretic peptide assay but not substantially interfering with the inactive natriuretic peptide assay;
determining kinetics of the administered therapeutic agent based on at least the active natriuretic peptide assay; and
determining cardiac diagnostic information based on at least the inactive natriuretic peptide assay.

2. The cardiac monitoring method as set forth in claim 1, wherein the assaying is performed after the administering.

3. The cardiac monitoring method as set forth in claim 1, wherein the assaying is performed both before and after the administering, and the determining of kinetics includes:
determining a difference between (i) the active natriuretic peptide assay performed after the administering and (ii) the active natriuretic peptide assay performed before the administering to determine uptake of the administered therapeutic agent.

4. The cardiac monitoring method as set forth in claim 1, wherein the assaying is performed a plurality of times, and the determining of kinetics includes:
determining a difference in the active natriuretic peptide assay performed the plurality of times, to determine uptake of the administered therapeutic agent.

5. The cardiac monitoring method as set forth in claim 1, wherein the inactive natriuretic peptide is NT-proBNP and the active natriuretic peptide is BNP.

6. The cardiac monitoring method as set forth in claim 5, wherein the administering of the therapeutic agent to the patient comprises:
administering nesiritide to the patient.

7. The cardiac monitoring method as set forth in claim 1, wherein the therapeutic agent includes a synthetic BNP, the active natriuretic peptide includes natural BNP produced in the patient responsive to cardiac stress, and the inactive natriuretic peptide includes at least one of NT-proANP, NT-proBNP, NT-proCNP, and NT-proDNP.

8. The cardiac monitoring method as set forth in claim 1, wherein a half-life of the inactive natriuretic peptide in the bloodstream is substantially longer than a half-life of the active natriuretic peptide in the bloodstream.

9. The cardiac monitoring method as set forth in claim 1, wherein a half-life of the inactive natriuretic peptide in the bloodstream is at least ten times longer than a half-life of the active natriuretic peptide in the bloodstream.

10. The cardiac monitoring method as set forth in claim 1, wherein the active natriuretic peptide and the inactive natriuretic peptide produced by a common biochemical pathway or a common precursor molecule.

11. A cardiac monitoring method comprising:
administering a therapeutic agent to a patient;
performing a first cardiac marker assay that is substantially affected by the therapeutic agent;
performing a second cardiac marker assay that is substantially not affected by the therapeutic agent;
determining kinetics of the administered therapeutic agent based on at least the first cardiac marker assay; and
assessing cardiac diagnostic information based on at least the second cardiac marker assay.

12. The cardiac monitoring method as set forth in claim 11, wherein the first cardiac marker includes a natural form of a first natriuretic peptide and the therapeutic agent includes a synthetic form or mimic of the first natriuretic peptide.

13. The cardiac monitoring method as set forth in claim 1t, wherein the therapeutic agent includes a synthetic or mimetic form of the first cardiac marker.

14. The cardiac monitoring method as set forth in claim 11, wherein the first cardiac marker includes a natural BNP, the second cardiac marker includes a natural NT-proBNP, and the therapeutic agent includes a synthetic BNP.

15. The cardiac monitoring method as set forth in claim 1, wherein the first cardiac marker and the second cardiac marker are produced by a common biochemical pathway or a common precursor molecule.

16. The cardiac monitoring method as set forth in claim 11, further including:
repeating the performing of the first cardiac marker assay at least once, the kinetics of the administered therapeutic agent being determined based on a change in the first cardiac marker between the repeated first cardiac marker assays.

17. The cardiac monitoring method as set forth in claim 11, wherein the administering of the therapeutic agent is performed a plurality of times over a treatment period, and the performing of the second cardiac marker assay comprises:
repeating the performing of the second cardiac marker assay a plurality of times over the treatment period, the cardiac diagnostic information being based on increase or decrease of the assayed second cardiac marker over the treatment period.

18. A cardiac monitoring apparatus for performing the cardiac monitoring method of claim 11.

19. A cardiac monitoring apparatus comprising:
a blood extraction device adapted to extract a plurality of blood samples from a patient undergoing treatment including administration of a synthetic or mimetic form of an active natriuretic peptide;
an assay device adapted to perform a blood assay yielding at least (i) an active natriuretic peptide level of a naturally produced natriuretic peptide and the synthetic or mimetic natriuretic peptide and (ii) an inactive natriuretic peptide level for each blood sample; and
an interpretation device outputting at least one of (i) kinetics of the administered synthetic or mimetic natriuretic peptide based on at least the first cardiac marker assay, and (ii) cardiac diagnosis information based on at least the second cardiac marker assay.

20. The cardiac monitoring apparatus as set forth in claim 19, wherein the active natriuretic peptide includes BNP, the inactive natriuretic peptide includes NT-proBNP, and the administered synthetic or mimetic natriuretic peptide includes a synthetic BNP.

21. The cardiac monitoring apparatus as set forth in claim 19, wherein the interpretation device comprises on of:
a card on which are recorded (i) level values of the blood assays and (ii) at least one computed value computed from one or more of the level values of one or more of the blood assays, and
an electronic spreadsheet in which are recorded level values of the blood assays, the electronic spreadsheet computing at least one computed value from one or more of the level values of one or more of the blood assays.
